(19) United States
(12) Patent Application Publication
(10) Pub. No.: US 2023/0172550 A1
(43) Pub. Date: Jun. 8, 2023

(54) Implantable ureteral stent and manufacture method thereof

(71) Applicant: Hydrustent, S.A, Barco Guimarães (PT)

(72) Inventors: Alexandre António ANTUNES BARROS, Caldas da Taipas (PT); Catarina PIRES SEPÚLVEDA, Braga (PT); Estevão Augusto RODRIGUES DE LIMA, Porto (PT); Pedro BRAGANÇA, Guimarães (PT); Rui Luís GONÇALVES DOS REIS, Porto (PT); Sara CARVALHEIRA NEVES, Braga (PT); Helena Filipa DU CUNHA FERNANDES, Vila Nova de Famalicão (PT); Hugo Filipe SIMÕES COSTA, Vila Nova de Famalicão (PT); André Filipe FERNANDES DA SILVA ANDRADE LEITE, Vila Nova de Famalicão (PT); Pedro Tiago DA CRUZ PEIXOTO, Vila Nova de Famalicão (PT); Bruno Guilherme GONÇALVES DE MATOS, Vila Nova de Famalicão (PT); Juliana Patrícia DA SILVA SOARES, Vila Nova de Famalicão (PT)

(21) Appl. No.: 17/995,755

(22) PCT Filed: Apr. 8, 2021
(86) PCT No.: PCT/IB2021/052929

§ 371 Date: Oct. 7, 2022

(30) Foreign Application Priority Data
Apr. 8, 2020 (EP) 20168857.9
Nov. 27, 2020 (PT) 116906

(51) Int. Cl. A61B 5/00; A61B 5/20

(52) U.S. Cl. CPC A61B 5/6862; A61B 5/205; A61B 5/6874; A61B 5/0031

(57) ABSTRACT

An implantable ureteral stent for implanting in the ureter comprising a first end for placing in the renal pelvis and a second end for placing in the bladder, each said end including a pressure sensor arranged to measure urinary pressure. Each pressure sensor can include an electronic circuit with electronic components and a substrate for receiving the electronic circuit and electronic components, wherein said substrate is a flexible membrane. The flexible membrane can be a sleeve surrounding the stent or the flexible membrane can be a flexible tube that is part of a thin tube that forms the stent, in particular the flexible membrane may have a thickness of 80-150 μm. The electronic components can be connected by wire-bonding. Each pressure sensor can have a flexible PCB having soldered electronic components. A manufacturing method is disclosed to make said implantable ureteral stent.

### TECHNICAL FIELD

The present disclosure relates to the field of implantable medical devices, more in particularly, the disclosure relates to a functionalized implantable ureteral stent.

### BACKGROUND

Alterations on the physiological parameters on the different regions of the urinary tract are often indicative of medical problems. The internal pressure is one of the essential parameters to be assessed in order to prevent and/or solve urinary complications.

The bladder is an organ essentially composed by epithelial tissue, muscle and connective tissue and it is neuro and anatomically related to the urethra and the urinary sphincters. These structures are responsible for chemical and mechanosensitive feedbacks. The urinary physiology allows the bladder to relax during filling (at low pressures) and can generate high pressures during voiding.

Several medical conditions can alter the vesical and sphincter physiology, thus interfering with the normal urinary function, such as the hyperactive bladder and prostate benign hyperplasia. These physiopathological alterations can translate into urinary disfunction and asymptomatic alterations. In any case, the involved structures as well as the ureters or the kidneys that depend on the normal bladder function can undergo irreparable damage due to high intravesical pressure. This is commonly associated to diseases derived from spinal cord injuries (SCI), Parkinson disease (PD), or multiple sclerosis (MS). These include neurogenic bladder (prevalence: SCI—70-84%, PD—55-80%, MS—50-75%) and associated complications such as vesical-ureteral reflux, with associated high risk of renal damage.

Additionally, the development of systems that allow the evaluation of the voiding pressure within the kidneys is fundamental, which is associated to a high risk of renal damage when the urine drainage is compromised (e.g. patients to whom the bladder was removed and an intestinal urinary derivation was performed, syndrome of pielo-ureteral junction, hydronephrosis, etc.).

The conditions justify the clinical need for the development of tools that allow fiduciary micturition evaluation.

The urodynamic test is probably the most used exam to evaluate the urinary function. This allows the evaluation of vesical pressures during the micturing cycle (from filling to voiding). It is performed in a clinical setting and requires the introduction of catheters with pressure sensors into the bladder, into an intra-abdominal organ (in order to evaluate the influence of the abdominal pressure on the vesical pressures) and at least a third catheter for the slow and progressive bladder filling with saline solution (in order to simulate the bladder filling for a posterior evaluation of the voiding phase). This test allows the measurement of the vesical pressures during the process.

So, urodynamic studies are a key component of the clinical evaluation of lower urinary tract dysfunction and include filling cystometry, pressure-flow studies, uroflowmetry, urethral function tests and electromyography. However, pitfalls of traditional urodynamics include physical and emotional discomfort, artificial test conditions with catheters and rapid retrograde filling of the bladder, which result in variable diagnostic accuracy.

Urodynamics test is the standard clinical procedure to evaluate the urinary tract function. However, its invasiveness and artificial character is evident: it does not simulate the patients' daily routine, and there is an associated physiological conditioning (it is performed in a clinical setting). Less invasive methods that allow the continuous and long-term monitoring of the urinary function in a more natural context are needed, since they would probably be better tolerated and more reliable.

Ambulatory urodynamic monitoring—AUM—uses physiological anterograde filling and, therefore, offers a longer and more physiologically relevant evaluation. However, AUM methods rely on traditional catheters and pressure transducers and do not measure volume continuously, which is required to provide context for pressure changes.

Thus, the urology solutions available on the market use:
equipment only available at the hospital;
catheters that are inserted into the patient at least into two natural orifices;
the urodynamics systems that state that are wireless refer to the communication of the catheter tips to the acquisition system.

Document US20190133472 relates to a system for monitoring a pressure in a biliary tract includes: a stent for a biliary tract including a pressure sensor; and a subcutaneous implant medical device including a communication module which receives a measured value of a pressure in a biliary tract through communication with the pressure sensor and a power module which supplies a power to the pressure sensor.

Document US20070027495A1 relates to o a sensor that is implantable to sense bladder condition. The disclosure describes an implantable bladder sensor that is attachable to an exterior surface of a urinary bladder to sense bladder condition or activity.

Other disclosed technologies describe:
wireless implantable devices designed for similar purpose of measuring the bladder pressure (but not kidney internal pressure);
catheters (non-wireless) with pressure sensors at the tip to measure intra-vesical and intra-renal pressures.

Besides the clear clinical advantages, the development of less invasive methods, compatible with continuous and long-term monitoring in a more natural context, may allow more accurate studies regarding the implications that pressure variations within the urinary tract may have on the different structures involved. Besides urodynamics, other clinical scenarios can benefit from this long-term and continuous monitoring. As example, the possibility to remotely control bleeding post-kidney transplant is an added value to the follow-up.

These facts are disclosed in order to illustrate the technical problem addressed by the present disclosure.

### General Description

An aspect of the present disclosure relates to an implantable ureteral stent for long-term implanting in the ureter comprising a first end for placing in the renal pelvis and a second end for placing in the bladder, each said end comprising a pressure sensor arranged to measure urinary pressure.

In an aspect, the sensors are configured to detect a differential intravesical pressure between the renal pelvis and the bladder.

Advantageously, the two sensors are electrically independent, that is, they are not connected electrically. This has benefits in the data acquisition process, reducing possible interferences and ensures that sensor's readings are totally electrically independent from each other. This also simplifies the stent construction, especially taking into account the distance between the two sensors.

The presence of a ureteral stent inside the urinary tract influences the pressure noticed along the ureter. The measurement of the pressure at bladder and kidneys should be as exact as possible, to correctly evaluate the condition of the stent and urinary tract where the stent is inserted. For safeguarding the urinary tract condition (including encrustations, incorrect placement of the stent, appropriate urine flow along the ureter, in particular within the stent and/or around the stent), it is fundamental to evaluate not only intra-renal and bladder pressures, but also the differential intravesical pressure between the renal pelvis and the bladder. Considering a long-term application, the stent should be a continuous tube that connects the kidney and the bladder along the ureter, promoting the drainage along the ureter, in particular within the stent and/or around the stent. If the drainage is compromised, complications may occur at kidney and bladder level, in particular at the kidney, due to an increase of pressure.

In an embodiment, each pressure sensor comprises an electronic circuit with electronic components and a substrate for receiving the electronic circuit and electronic components, wherein said substrate is a flexible membrane.

In an embodiment, the flexible membrane is a sleeve surrounding the stent or the flexible membrane is a flexible tube that is part of a thin tube that forms the stent, in particular the flexible membrane having a thickness of 80-150 μm.

The electronic components of the implantable ureteral stent, as described in previous embodiments, are connected by wire-bonding.

In an embodiment, each pressure sensor comprises a flexible printed circuit board (PCB) having soldered electronic components.

In an embodiment, one or more of said sensors comprises an antenna for receiving power wirelessly.

In an embodiment, one or more of said sensors comprises an antenna for transmitting data wirelessly.

In an embodiment, the antenna is a near-field communication, NFC, antenna.

In an embodiment, the implantable ureteral stent of the present disclosure may comprise a liquid-tight encapsulation of said pressure sensors.

In an embodiment, the implantable ureteral stent of the present disclosure may have a diameter inferior to 3 mm, preferably 2.5-2 mm.

In an embodiment, the pressure sensor to be placed in the kidney is configured to detect a relative pressure of 0 up to 200 cm H₂O (19.6 kPa).

In an embodiment, the pressure sensor to be placed in the bladder is configured to detect a relative pressure of 0 up to 100 cmH₂O (9.8 kPa).

In an embodiment, the sensors are configured to detect a differential intravesical pressure between renal pelvis and bladder up to 200 cmH₂O (19.6 kPa).

In an embodiment, the transmitter comprises an antenna that comprises an operation frequency of 6-60 MHz, in particular 13.56 MHz.

In an embodiment, each sensor has an elongated antenna arranged longitudinally along the stent.

In an embodiment, each sensor comprises two antennas placed diametrically opposite in respect of the stent. In particular, a first antenna for receiving power wirelessly and a second antenna for transmitting data wirelessly, preferably both antennas being NFC-frequency antennas.

In an embodiment, the plurality of pressure sensors is selected from capacitive sensor, piezoresistive sensor, or combinations thereof.

In an embodiment, the implantable ureteral stent of the present disclosure may further comprise a pH sensor, a temperature sensor, a flow sensor, a volume sensor, or combinations thereof.

In an embodiment, the implantable ureteral stent of the present disclosure may further comprise an electronic data processor arranged to detect and calculate intravesical pressure during micturition (for example, when pressure starts to decrease, signaling the start of micturition) and/or during bladder filling (for example, when pressure starts to increase, signaling the end of micturition)—that is, the electronic data processor may be arranged to detect and calculate intravesical pressure during micturition, or may be arranged to detect and calculate intravesical pressure during bladder filling, or may be arranged to detect and calculate intravesical pressure whenever any micturition or bladder filling is detected.

In an embodiment, the electronic data processor is arranged to calculate the differential intravesical pressure between renal pelvis and bladder.

The sensors may be applied to the external surface of the stent, to measure the pressure of the organ where they are placed.

It is also described a kit comprising the implantable ureteral stent according to any of the disclosed embodiments and an external reader comprising an antenna and an electronic circuit for communicating wirelessly with said stent.

Another aspect of the present disclosure relates to a manufacture method for providing an implantable ureteral stent of the present disclosure, comprising the steps of:
providing a ureteral stent for implanting in the ureter having a first end for placing in the renal pelvis and a second end for placing in the bladder;
providing each said end with a pressure sensor arranged to measure urinary pressure.

### BRIEF DESCRIPTION OF THE DRAWINGS

The following figures provide preferred embodiments for illustrating the description and should not be seen as limiting the scope of invention.

FIG. 1 shows a schematic representation of an embodiment of a wireless system to monitor the urinary function implantable ureteral stent 3 integrating a wireless electronic component 7 that may comprise a sensor, an interface and a transmitter to monitor the urinary function. The system is placed internally in the ureter 1, as the upper ureter 4, between the kidney 2 (in the renal pelvis 5) and the bladder 6. The electronic component 7 may contain a transmitter receiving energy from a mobile wireless device 8 and transmitting it to the remaining components.

FIG. 2 shows a schematic representation of an embodiment of the interface system of the implantable ureteral stent of the present disclosure that establishes the communication between the transmitter and the sensor. The sensor may receive the energy and then captures and transmits the pressure data acquired back to the interface. On its turn, the interface passes the sensor information to the transmitter. The transmitter emits the collected data via the antenna to a wireless device 8. The wireless device 8 functions both as energy emitter and data receiver/analyzer.

FIG. 3 shows a schematic representation of an embodiment of the interface system of the implantable ureteral stent of the present disclosure that establishes the communication between NFC transmitter and sensor.

FIG. 4 depicts a schematic drawing of an embodiment of the device to be integrated on the stent comprising a pressure sensor 9, an analog-to-digital converter 10, a microcontroller unit 11, a wireless transceiver 12 and a wireless antenna 13, in particular the wireless communications being NFC.

FIG. 5 depicts an embodiment of the arrangement of the NFC antennas on the stent.

FIG. 6 is a schematic representation of an embodiment of the external reader, comprising an NFC initiator/HF Reader 14, a microcontroller unit 15, Bluetooth 16, USB connector 17, rechargeable battery 18, charge management circuit 19, and an NFC reader antenna 20.

FIG. 7 is a schematic representation of an embodiment of the device coupled to a ureteral stent, showing the PCB electronic system 21, the stent antenna 13, and the implantable ureteral stent 3.

FIG. 8 is a schematic representation of the stents' cross-section in the electronic component region, showing the placement of the electronic component 7, and the PCB 21 on the ureteral stent 3. An internal coating/sleeve 22 is placed between the ureteral stent 3 and the PCB 21. This internal coating/sleeve may cover all the stent or only part of it. The electronic component and the PCB are covered by an external coating/sleeve 23, that can be designed to cover all the stent, or only a part of it, providing that the electronic component and the PCB are covered by it.

FIG. 9 schematically shows different embodiments of ureteral stents and the placement of the electronic components 7, for polymeric or metallic stent embodiments.

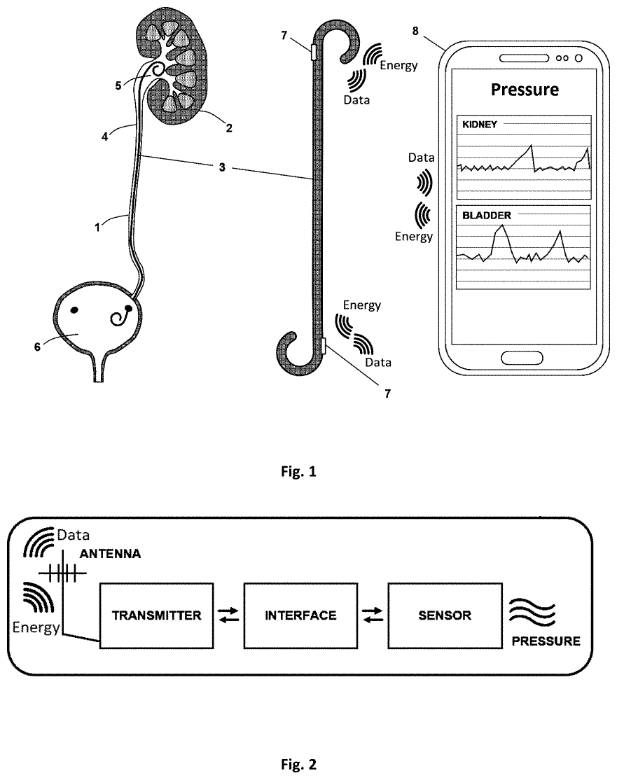

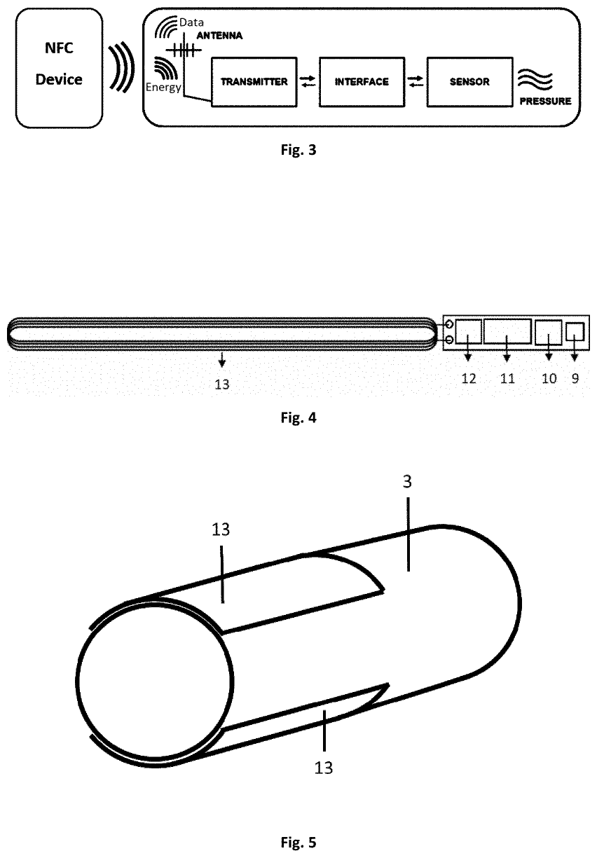

### DETAILED DESCRIPTION

The present disclosure relates to an implantable ureteral stent for implanting in the ureter comprising a first end for placing in the renal pelvis and a second end for placing in the bladder, each said end comprising a pressure sensor arranged to measure urinary pressure. In an embodiment, the sensors are configured to detect a differential intravesical pressure between the renal pelvis and the bladder. Each pressure sensor may comprise an electronic circuit with electronic components and a substrate for receiving the electronic circuit and electronic components, wherein said substrate is a flexible membrane. The flexible membrane may be a sleeve surrounding the stent or the flexible membrane may be a flexible tube that is part of a thin tube that forms the stent, in particular the flexible membrane may have a thickness of 80-150 μm. The electronic components may be connected by wire-bonding. Each pressure sensor may comprise a flexible PCB having soldered electronic components. A manufacture method for providing said implantable ureteral stent is also disclosed.

An aspect of the present disclosure relates to an implantable ureteral stent 3 for implanting in the ureter 1 comprising a first end for placing in the renal pelvis 5 and a second end for placing in the bladder 6, each said end comprising a pressure sensor 7 arranged to measure urinary pressure. In an aspect, the sensors are configured to detect a differential intravesical pressure between the renal pelvis (5) and the bladder (6).

In an embodiment, each pressure sensor 7 comprises an electronic circuit with electronic components and a substrate for receiving the electronic circuit and electronic components, wherein said substrate is a flexible membrane.

The electronic components of the implantable ureteral stent 3, as described in previous embodiments, are connected by wire-bonding.

In an embodiment, one or more of said sensors 7 comprises an antenna for receiving power wirelessly.

In an embodiment, one or more of said sensors 7 comprises an antenna for transmitting data wirelessly.

In an embodiment, the implantable ureteral stent of the present disclosure may comprise a liquid-tight encapsulation of said pressure sensors 7.

In an embodiment, the sensors are configured to detect a differential intravesical pressure between renal pelvis 5 and bladder 6 up to 200 cmH₂O (19.6 kPa).

In an embodiment, the electronic data processor is arranged to calculate the differential intravesical pressure between renal pelvis 5 and bladder 6.

Another aspect of the present disclosure relates to a manufacture method for providing an implantable ureteral stent 3 of the present disclosure, comprising the steps of:
providing a ureteral stent 3 for implanting in the ureter 1 having a first end for placing in the renal pelvis 5 and a second end for placing in the bladder 6;
providing each said end with a pressure sensor 7 arranged to measure urinary pressure.

In an embodiment, the implantable ureteral stent of the present disclosure is a portable homecare monitoring solution for urinary pressure.

In an embodiment, the sensor's data is collected externally, transmitted via the NFC antenna, and monitored in a portable device (smartphone, tablet, etc.) using a dedicated application.

In an embodiment, the implantable ureteral stent of the present disclosure comprises at least two pressure sensors, an interface for example a microcontroller, and a transmitter.

In an embodiment, the implantable ureteral stent of the present disclosure comprises a urological stent integrated with wireless pressure sensors and near-field communication (NFC) antenna. Alternatively, other wireless short-range communications may be used, for example a body area network communication, or a Bluetooth connection, or a communication in UHF, Ultra High Frequency.

In an embodiment, the pressure sensor is selected from a list of capacitive sensors, piezoresistive sensor, or combinations thereof.

In an embodiment, the pressure sensor is wireless (based on microelectromechanical system, MEMS) and the antenna is a near-field communication (NFC) antenna.

In an embodiment, the system is battery-free, and therefore the system harvests energy provided by and external power source.

In an embodiment, a power emitting source provides energy wirelessly via the NFC antenna.

In an embodiment, the MEMS system is then able to measure the pressure values and sends back this information.

In an embodiment, the external power source may be a portable device such as a mobile phone or a computer, in particular that also collects and analyses the data.

In an embodiment, the electronic components are assembled to the stent using an adhesive/glue, optionally a cured adhesive/glue, optionally by thermal or UV treatment. The assembly is then coated/encapsulated with a biocompatible coating.

In an embodiment, the implantable ureteral stent of the present disclosure may further comprise a pH sensor, temperature sensor, flow sensor, volume sensor, or combinations thereof, among others.

In an embodiment, the implantable ureteral stent of the present disclosure can be divided in two main components: the ureteral stent and the electronic component.

In an embodiment, the implantable ureteral stent of the present disclosure may comprise silicone, polyurethane, or mixtures thereof, among others.

In an embodiment, the electronic component may comprise the different sub-components, that are encapsulated and isolated from the surrounding environment using a biocompatible resin.

In an embodiment, the implantable ureteral stent of the present disclosure comprises a pressure sensor that acquires and emits the recorded data wirelessly.

In an embodiment, the disclosure has a pressure sensor, or sensors, that acquires and emits the recorded data wirelessly. Current methods use pressure sensors connected (via wires) to external devices.

Some current solutions do not simulate the patient's daily routine and do not allow the patient to go home and monitor the intra-urinary pressures. Also, some current solutions do not measure the intra-renal pressure and there is an associated physiological conditioning (it is performed in a clinical setting).

Bearing in mind that the stent will be placed in the urinary tract in the long term, the diameter of the sensor-stent assembly should be of 3 mm or less. Also, the sensor-stent assembly does not have an energy storage unit, being then necessary to select low energy consumption components.

FIG. 4 shows an embodiment of the device to be integrated on the stent.

The present disclosure is more particularly described in the following example that is intended as illustrative only since numerous modifications and variations are possible and will be apparent to those skilled in the art.

In an embodiment, it was selected the Murata's capacitive absolute pressure sensor SCB10H 9. This family of sensors has a wide range of pressures, being 8012 the most interesting series, since it works for a range between 0 and 1220 cmH₂O. In addition, it is a capacitive sensor based on MEMS that is not encapsulated, allowing a customized coupling for the application intended in this disclosure. The high resistance and low passive capacitance insulation of this sensor allows for very low energy consumption, high stability and accuracy over time and temperature variations. That is advantageous since measurements cannot be affected by external factors such as humidity, temperature and mechanical or chemical shocks and it will have to last for at least the lifetime of the implanted ureteric stent. Additionally, this sensor will be encapsulated with a thin biocompatible silicone.

The analog-to-digital signal converter 10, in addition to being specific to the sensor that has been selected, it meets certain requirements, such as dimensions, power consumption, operating range and interface. In an embodiment, it was used a Renesas' ZSSC3123, a CMOS integrated circuit for precise conversion of capacitances into digital signal and specific signal correction from capacitive sensors. The digital compensation of the sensor offset, sensitivity and temperature deviation are performed by means of an internal digital signal processor executing a correction algorithm with calibration coefficients stored in a non-volatile EEPROM. The data acquired and corrected by this component are sent to the microcontroller (master) 11, that manages the communication through a Serial Peripheral Interface (SPI) protocol. The microcontroller is also responsible for controlling communication with the wireless transmitter by an I2C digital interface.

Whenever the system receives energy, it starts to acquire data from the pressure sensor, which is processed and filtered on a first stage through the interface between the sensor and the transmitter. The microcontroller is also responsible for controlling the communication with the wireless transmitter. In an embodiment, a STM8 microcontroller from STMicroelectronics was selected, essentially due to its dimensions, low consumption and compatible communication interface with the transmitter (I2C).

Connected to the microcontroller is a radio-frequency identification (RFID) transmitter, the integrated circuit (IC) responsible for sending data to an exterior receiver device. Communication between the stent and the exterior reader is established through NFC technology, at a frequency of 13.56 MHz. Due to the frequency of operation and communication protocol used, it is necessary that this controller supports the specifications established for NFC communication. In an embodiment, a STMicroelectronics controller ST25DV04K was selected as NFC transceiver 12. Another main functionality of this component is the possibility to harvest electromagnetic energy through the antenna attached to it. In this way, it can not only supply its own internal circuit, but also supply power to external components, such as the microcontroller and the sensor. It is therefore, an indispensable component of this non-invasive wireless sensor communication system. This is physically connected to the microcontroller through a digital interface (I2C) and endows the wireless communication system with the external reader through the physical connection of an antenna.

In an embodiment, the loop antenna 13 is based on a 0.1 mm copper wire coil, fabricated on a flexible substrate. It has preferably a length and width of 6 cm and 0.25 cm, respectively, with an inductance less than or equal to 4.83 μH. It is connected to the terminals of the ST25DV04K. The NFC antenna-controller is compensated with an external synchronization capacitor in the pF range to improve the system's range.

In an embodiment, each sensor has an elongated antenna arranged longitudinally along the stent.

In an embodiment, each sensor comprises two antennas placed diametrically opposite in respect of the stent (FIG. 5). In particular, a first antenna for receiving power wirelessly and a second antenna for transmitting data wirelessly, preferably both antennas being NFC-frequency antennas.

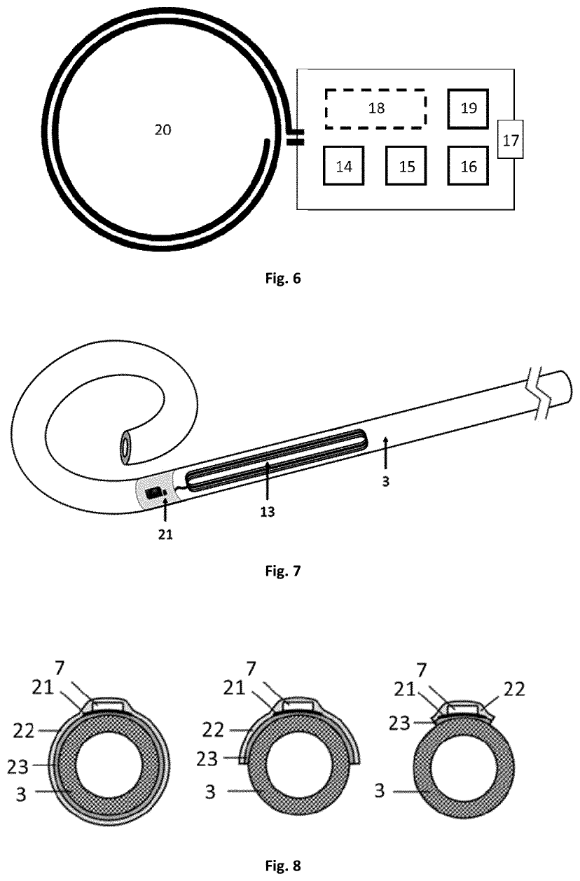

This solution includes the development of a mobile data acquisition device and a smartphone application to visualize the data collected from the stent. Thus, it is necessary to have a device that works as an NFC receiver, schematically represented in FIG. 6. In an embodiment, the adopted approach involved the development of a mobile NFC reader, powered by a rechargeable battery, capable of communicating via Bluetooth with the user's smartphone, presenting the data obtained through an app. The advantage of this option is that it allows to extend the reading range when compared to the NFC system integrated in the mobile phone.

On the present embodiment, the NFC Initiator/HF Reader 14 is a highly integrated IC, including the analog front end (AFE) and a highly integrated data framing system for ISO 18092 (NFCIP-1) initiator, ISO 18092 (NFCIP-1) active target, ISO 14443A and B reader (including high bit rates), ISO 15693 reader and FeliCa™ reader. It is intended to directly drive external antennas, and to detect transponder modulation superimposed on the 13.56 MHz carrier signal. A 4-wire Serial Peripheral Interface (SPI) is used for communication between the external microcontroller and the IC.

A microcontroller unit 15 manages the communication between the reader and the Bluetooth. It is connected with the IC reader through a SPI digital interface and with the Bluetooth by universal asynchronous receiver transmitter (UART) interface. By its turn, Bluetooth 16 transmits the data collected from de pressure sensor to the user's smartphone. As previously mentioned, it is connected to the microcontroller through an UART interface.

A USB connector 17 is used to charge, by cable, a rechargeable battery 18 that powers the wireless reader device. A charge management circuit 19, a battery charge management system, integrates the most common functions for wearable and portable devices, namely a charger, a regulated output voltage rail for system power, and ADC for battery and system monitoring. It integrates advanced power path management and control that allows the device to provide power to the system while charging the battery.

The NFC reader antenna 20 of the external reader device is a loop antenna fabricated on a flexible substrate with 15 cm diameter has an inductance of approximately 1 μH. The NFC antenna resonance frequency is adjusted with external tuning capacitors.

On second embodiment, a method of wireless energy transfer was considered to supply the stent. A wireless transmitter can induce energy to supply the electronic circuit, through an antenna integrated in the stent and a resonant circuit. Thus, the supply circuit has a wireless interface with the exterior, based on energy transfer by induction. This modular wireless energy interface supplies the acquisition system without using an energy harvesting NFC transceiver. It allows the use of a different wireless communication technology (such as Bluetooth, Wi-Fi, radiofrequency, ZigBee, etc.) and, therefore, improve the communication range of the implanted device. In this solution, the antenna used to transmit the acquired data is independent from the antenna used on the energy harvesting circuit.

To transmit data using Bluetooth technology, the DA14531 ultra-low power system-on-a-chip (SoC) integrating a 2.4 GHz transceiver from Dialog Semiconductor was selected. It can be used as a standalone application processor or as a data pump in hosted systems and is compatible with Bluetooth V5.1, ETSI EN 300 328 and EN 300 440 Class 2 (Europe), with a typical range of up to 10 meters. The antenna is a commercially available 2.4 GHz chip antenna from Johanson Technology with 0.37 mm thickness. With this solution, the data is directly transmitted to the patient's smartphone.

Miniaturization of the circuit is achieved by the development of thin and flexible PCBs, assembling the micro components on its surface. This flexible PCB is fixed on the stent (FIG. 7) using a biocompatible epoxy and a thin heat shrink tubing, covering the entire system except for the pressure sensor.

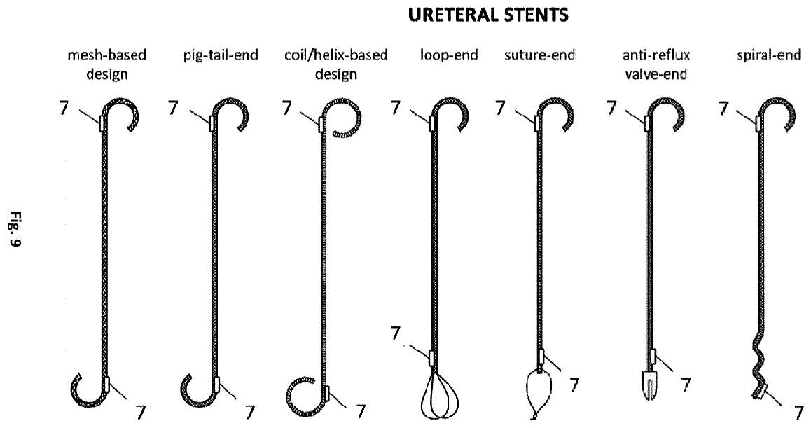

After connecting the components and fixing the device to the stent, the circuit is encapsulated with a biocompatible surface coating material that allows the proper protection of the sensors without interfering with the measurement of physiological parameters (FIG. 8).

Respecting the requirements mentioned above, the correct functioning of the system is promoted, as well as the user's comfort, also reducing the possibility of acute reactions to the implantation of the device.

The term “comprising” whenever used in this document is intended to indicate the presence of stated features, integers, steps, components, but not to preclude the presence or addition of one or more other features, integers, steps, components or groups thereof. The disclosure should not be seen in any way restricted to the embodiments described and a person with ordinary skill in the art will foresee many possibilities to modifications thereof. The above-described embodiments are combinable. The following claims further set out particular embodiments of the disclosure.

1. An implantable ureteral stent for long-term implanting in the ureter comprising a first end for placing in the renal pelvis and a second end for placing in the bladder, each said end comprising a pressure sensor arranged to measure urinary pressure, wherein the sensors are configured to detect a differential intravesical pressure between the renal pelvis and the bladder.

2. The implantable ureteral stent according to claim 1, further comprising an electronic data processor arranged to detect and calculate pressure during micturition and/or during bladder filling.

3. The implantable ureteral stent according to claim 1, further comprising an electronic data processor arranged to calculate a differential pressure between the renal pelvis sensor and the bladder sensor for obtaining a differential intravesical pressure between renal pelvis and bladder.

4. The implantable ureteral stent according to claim 1, wherein the sensors are configured to detect a differential intravesical pressure between the renal pelvis and the bladder up to 200 cmH₂O (19.6 kPa).

5. The implantable ureteral stent according to claim 4, wherein the pressure sensor to be placed in the kidney is configured to detect a relative pressure of 0 up to 200 cmH₂O (19.6 kPa).

6. The implantable ureteral stent according to claim 1, wherein the pressure sensor to be placed in the bladder is configured to detect a relative pressure of 0 up to 100 cmH₂O (9.8 kPa).

7. The implantable ureteral stent according to claim 1, wherein each pressure sensor comprises an electronic circuit with electronic components and a substrate for receiving the electronic circuit and electronic components, wherein said substrate is a flexible membrane.

8. The implantable ureteral stent according to claim 1, wherein the flexible membrane is either (i) a flexible tube that is part of a thin tube that forms the stent, or (ii) a sleeve surrounding the stent and having a thickness of 80-150 μm.

9. The implantable ureteral stent according to claim 1, wherein the two sensors are electrically independent.

10. The implantable ureteral stent according to claim 1, wherein each pressure sensor comprises a flexible PCB having soldered electronic components.

11. The implantable ureteral stent according to claim 1, wherein one or more of said sensors comprises an antenna for receiving power wirelessly.

12. The implantable ureteral stent according to claim 1, wherein one or more of said sensors comprises an antenna for transmitting data wirelessly.

13. The implantable ureteral stent according to claim 1, further comprising a transmitter with an antenna that comprises an operation frequency of 6-60 MHz.

14. The implantable ureteral stent according to claim 1, wherein the stent has a diameter inferior to 3 mm.

15. The implantable ureteral stent according to claim 1, wherein the plurality of pressure sensors is selected from capacitive sensor, piezoresistive sensor, or combinations thereof.

16. The implantable ureteral stent according to claim 1, further comprising a pH sensor, a temperature sensor, a flow sensor, a volume sensor, or combinations thereof.

17. A manufacture method for providing an implantable ureteral stent, comprising the steps of:
providing a ureteral stent for implanting in the ureter having a first end for placing in the renal pelvis and a second end for placing in the bladder;
providing each said end with a pressure sensor arranged to measure urinary pressure.
